(19) United States
(12) Patent Application Publication
(10) Pub. No.: US 2025/0154413 A1
(43) Pub. Date: May 15, 2025

(54) LABORATORY GRAM-SCALE PYROLYSIS REACTOR

(71) Applicant: RIJKSUNIVERSITEIT GRONINGEN, Groningen (NL)

(72) Inventors: Songbo HE, Groningen (NL); Hero Jan HEERES, Groningen (NL); Jessi OSORIO VELASCO, Gronigen (NL)

(21) Appl. No.: 18/838,191

(22) PCT Filed: Feb. 21, 2023
(86) PCT No.: PCT/EP2023/054302

§ 371 Date: Aug. 13, 2024

(30) Foreign Application Priority Data
Feb. 21, 2022 (NL) 2031021

(51) Int. Cl. C10B 53/02; C10B 53/07; G01N 33/00

(52) U.S. Cl. CPC C10B 53/02; C10B 53/07; G01N 33/0009

(57) ABSTRACT

The invention relates to an apparatus and method for pyrolyzing a feedstock in an open container. The apparatus comprises a downer reactor with an entry located at the top end of the downer for feeding and an exit located at the bottom end of the downer for removing the container from the downer, the downer being divided into at least three vertically arranged zones by at least two locking systems on either side of a middle pyrolysis zone, wherein the at least two locking systems are configured to simultaneously close for sealing the pyrolysis zone, wherein the at least two locking systems are further configured to let through the container in an open position, the apparatus comprising heating means, cooling means and pressurizing means configured to adjust the temperature and pressure of at least the pyrolysis zone independently from the other zones.

The invention relates to an apparatus and method for pyrolyzing a feedstock in an open container.

### BACKGROUND ART

Pyrolysis of solids such as lignocellulosic biomass or plastics in a pyrolysis reactor and particularly in a pressurised pyrolysis reactor, such as in the case of hydropyrolysis, often requires dedicated feeding equipment such as screw conveyors. This is difficult to realize for laboratory gram-scale (i.e. between 1-30 g/h solid feeding) research equipment due to the very low feeding rate. Presently available laboratory-scale pyrolysis equipment (e.g. Pyroprobe 5200 or tandem microreactor TMR Rx-3050TR) only allows experiments on a microgram scale in a batch-wise operation and does not provide possibilities for the collection of all of the products including gas, liquid, and solids.

Ellens et al., in Bioresource Technology 103 (2012) 374-380, disclosed a vertical stainless steel reactor, which is a 0.021 m diameter pipe with 1.8 m of heated length. The length is dictated by the time required to pyrolyze biomass particles with an average diameter of 400 μm in a free-fall mode. Four ceramic heater sections enclose the pipe and radiatively heat the reactor. A sealed volumetric feeder meters biomass into the top of the reactor. A mass flow controller regulates nitrogen gas into the reactor. Most of the char produced is collected in a stainless steel char catch. Pyrolysis vapors are captured by condensers, an electrostatic precipitator and ice bath. Non-condensable gases leave through the gas outlet, and are analysed by a gas chromatograph. Residence time of the biomass is dictated by the length of the tube. There is no section for catalytic upgrading the pyrolysis vapors, and the reactor cannot be operated under a high pressure.

Chen et al., in Energy Conversion and Management 220 (2020) 113065 disclosed a lab-scale semi-continuous free-fall pyrolysis unit suitable for rapid testing of a wide range of feedstocks, operating conditions (pressure and temperature), carrier gases (a.o. hydrogen, nitrogen), and optionally a wide range of catalysts. The integrated reactor is composed of an upper drop-tube pyrolysis section and a lower moving-bed upgrading section. The unit allows introducing a (biomass) source in the pyrolysis reactor without feeding issues. Furthermore, the unit allows the continuous operation and also the collection of all product phases, which can be used to determine the mass balances. The downer is 7-meter long. The reactor cannot be operated under a high pressure.

WO 2020/212198 A1 discloses a continuous reactor for treatment of biomass. The reactor comprises a plurality of separate compartments: a drying compartment, a torrefaction compartment and a pyrolysis compartment. The continuous reactor further comprises a transport device for transportation of the biomass through the compartments. Such a transport device comprises moving parts which are subject to dysfunction caused by contamination and wear.

It is an objective of the present invention to overcome at least one of the abovementioned drawbacks or at least to provide a useful alternative. It is another objective of the present invention to provide an apparatus for pyrolyzing a feedstock, to which even cumbersome feedstocks such as lignin can be fed without difficulty. It is another objective of the present invention to provide an apparatus for pyrolyzing a feedstock which will fit in a research laboratory. It is another objective of the present invention to provide an apparatus for pyrolyzing a feedstock which can perform hydropyrolysis. It is another objective of the present invention to provide an apparatus for pyrolyzing a feedstock from which all product phases can be collected. It is another objective of the present invention to provide an apparatus for pyrolyzing a feedstock which can be operated semi-continuously with respect to the solid feed. It is another objective of the present invention to provide an apparatus for pyrolyzing a feedstock which has the option of catalytic upgrading of the products. It is a further objective of the present invention to provide an apparatus for pyrolyzing a feedstock which provides for rapid testing of a wide range of feedstocks, operating conditions (pressure, temperature, and residence time), carrier gases (a.o. hydrogen, nitrogen, methane, ethylene, product recycle gas, etc.), and optionally a wide range of catalysts. It is a further objective of the present invention to provide an apparatus for pyrolyzing a feedstock in which the feedstock may be liquefied in a reaction medium/catalyst at a relatively low temperature before being pyrolyzed.

Thereto, the present invention provides an apparatus for pyrolyzing a feedstock in an open container, the apparatus comprising a downer with an entry for feeding and an exit for removing the container from the downer, the downer being divided into at least three zones by at least two locking systems on either side of a middle pyrolysis zone, wherein the at least two locking systems are configured to simultaneously close for sealing the pyrolysis zone, wherein the at least two locking systems are further configured to let through the container in an open position, the apparatus comprising heating means, cooling means, and pressurizing means configured to adjust the temperature and pressure of at least the pyrolysis zone independently from the other zones.

The invention also provides a method for pyrolyzing a feedstock in an open container, the method comprising:
a) loading the feedstock into the open container,
b) placing the container in a pre-treatment zone,
c) pre-heating the feedstock, preferably to a temperature of 50-250° C.,
d) transferring the container to a pyrolysis zone,
e) pyrolyzing the feedstock, preferably at a temperature of 400-450° C. and a pressure of 1-60 bar,
f) transferring the container to a cooling zone and allowing the feedstock to cool, preferably to a temperature of below 50° C.

By placing the feedstock in an open container, and sequentially leading the container through different zones, in which zones the conditions can independently be adjusted, the pyrolysis process is simplified and more easily adjustable as compared to pyrolysis processes in traditional free-fall reactors in which residence times are dictated by the length of the free-fall tube. Furthermore, the whole sequence of leading a container through different zones can be repeated successively, leading to semi-continuous loading of the solid feed. This leads to a highly versatile apparatus and process.

The open container may be any type of open container which is resistant to pyrolysis temperatures, e.g. up to 500° C. With the term “open” is meant that at least part of the container is permeable to pyrolysis vapors. The feedstock is to be loadable to the container through an opening, which opening may optionally be closable or sealable. For example, the open container may be a simple quartz tube which optionally comprises a cap. In case the container comprises a cap, then the cap or at least part of the container is permeable to gases such as pyrolysis vapors. For example, the cap or the container itself may comprise holes, pores, a mesh or the like, through which the pyrolysis vapors may exit the container, but through which the solid residue such as feedstock and char may not exit the container. Preferably the open container is a quartz test tube.

A downer or downer reactor is a reactor through which the feedstock to be reacted, in the case of the present invention: pyrolyzed, moves in a downward fashion. Although such a reactor may in principle have any type of shape, a tubular shape is most common and also highly preferred.

In the downer, the at least three different zones are therefore arranged vertically. The entry is located at the top end of the downer, whereas the exit is located at the bottom end of the downer. The vertical arrangement allows the open container to pass through the different zones by the influence of gravity when the locking systems are in an open position. For example, when the locking system between two zones is changed from a closed to an open position, the open container may fall from the upstream zone to the downstream zone underneath. Under normal operating conditions a locking system at the entry of a zone will be in the open position and a locking system at the exit of a zone (and/or entry of a downstream zone of the concerned zone) will be in the closed position for a container to be placed in the concerned zone. When then closing the locking system at the entry of the concerned zone, the container is sealed from the other zones and/or the environment.

Thus, the locking systems on either end (upstream and downstream) of a zone define a pair of locking systems, which are configured to simultaneously close for sealing a zone when both locking systems are in a closed position. The locking systems at the exit of a zone may then independently be opened for transferring the container from this zone to a downstream zone.

The locking systems, preferably at least the pre-treatment zone locking system, pyrolysis zone entry locking system, and pyrolysis zone exit locking system, preferably each comprise a temperature and pressure resistant valve. Preferably, the valves are pressure resistant to a pressure of at least 100 bar and temperature resistant up to a temperature of at least 450° C. Valves may be any type of valve suitable for letting through a container, such as ball valves or slide valves.

The apparatus is suitable for pyrolyzing feedstock on a gram-scale (i.e. 1-100 g, preferably 1-30 g). As will be understood by the skilled person, the mentioned gram-scale relates to the amount of feedstock that may simultaneously be pyrolyzed in the pyrolysis zone. Therefore, the open container has a volume of about 1-120 ml, preferably 1-40 ml. For example, the open container may be cylindrical container with a diameter of 0.5-4 cm, preferably of 1-3 cm, and a height of 5-50 cm, preferably of 20-40 cm. In order to facilitate transport of such open containers through the downer and the pyrolysis of the feedstock therein, the downer preferably has an inner diameter in the range of between 1-5 cm, preferably 2-4 cm. Preferably, the container has a ratio of length: diameter of about 1:15 to 1:20. The outer diameter of the container is preferably about 2-4 mm smaller than the inner diameter of the downer.

The apparatus comprises heating means, cooling means, and pressurizing means configured to adjust the temperature and pressure of at least the pyrolysis zone independently from the other zones, to a pyrolysis temperature and a pyrolysis pressure. Preferably, the apparatus comprises heating means, cooling means, and pressurizing means configured to adjust the temperature and/or pressure of all zones independently from the other zones. For example, the heating means may be an electronic heating jacket enclosing the pyrolysis zone. The cooling means may be a circulating liquid such as ethanol or water. The circulating liquid preferably has a temperature of −30-1° C. The pressurizing means may for example comprise a back pressure regulator (BPR) which controls/maintains pressure from a high pressure-gas cylinder.

Preferably, the apparatus comprises a pyrolysis carrier gas inlet and a pyrolysis vapor outlet, both in fluid communication with the pyrolysis zone. The pyrolysis carrier gas inlet is advantageously in fluid communication with a carrier gas source, such as one or more gas cylinders, for feeding carrier gas to the reactor. The pyrolysis carrier gas may for example be N₂, H₂, recycled pyrolysis gas, or a mixture of one or more of these gases.

Preferably, the apparatus comprises a pre-heater in fluid communication with the pyrolysis carrier gas inlet. The pre-heater advantageously pre-heats the carrier gas, such that the carrier gas entering the pyrolysis zone has a temperature close to the pyrolysis temperature in the pyrolysis zone. This prevents temperature gradients and results in more predictable and repeatable results for better analysis of the pyrolysis process. Preferably, the pre-heater heats the pyrolysis carrier gas to a temperature of not more than 10° C. lower than the pyrolysis temperature and not more than 10° C. higher than the pyrolysis temperature.

Preferably, the apparatus further comprises a catalytic upgrading reactor having a gas inlet in fluid communication with the pyrolysis vapors outlet. Accordingly, pyrolysis vapors are then transported out of the pyrolysis zone and into the catalytic upgrading reactor by the pyrolysis carrier gas.

The catalytic upgrading reactor may be a moving bed reactor, fluidized bed reactor, circulating fluidized bed reactor or a fixed-bed reactor. Advantageously, the catalytic upgrading reactor is a catalytic fixed-bed reactor. The catalytic fixed-bed reactor may be a cylindrical tube filled with catalysts, preferably catalyst particles, such as catalyst pellets. The catalyst particles are held in a fixed position while reactants, in this case the pyrolysis vapors carried by the pyrolysis carrier gas are flowing through the bed and are converted into catalytic pyrolysis products. The catalyst may have multiple configurations including one relatively large bed, several horizontal beds, several parallel packed tubes, or multiple beds in their own shells. The catalyst may be any type of catalyst suitable for catalytic upgrading of pyrolysis vapors, examples of such catalysts are zeolites, supported metal catalysts or any other suitable industrial catalysts with a particle size of about 0.2-3 mm. For research purposes, the catalytic upgrading reactor and/or the catalytic fixed bed therein are replaceable. For example, the catalytic fixed-bed reactor may be connected to the pyrolysis vapor outlet and a further element of the apparatus, such as the inlet of a cooling unit, with easy connecting means, such as union fittings, e.g. provided by Swagelok.

Preferably, the catalytic upgrading reactor comprises a gas outlet in fluid communication with the inlet of a cooling unit. The cooling unit cools the pyrolysis vapor to liquid pyrolysis products and incondensable gases. The cooling unit may for example comprise at least one condenser, and preferably several condensers in series, wherein the gas outlet of one condenser is connected to the gas inlet of a subsequent condenser. Each condenser captures its own fraction of liquid pyrolysis product. The gas outlet of the final, i.e. most downstream condenser, is advantageously connected to an analyzer, such as a gas chromatograph. The analyzer then serves to analyze the incondensable gases.

Thus, in a preferred embodiment, the apparatus comprises an analyser for analysing pyrolysis gas products. The analyzer is preferably in fluid connection with the gas outlet of the final condenser.

Preferably, the closed pyrolysis zone is pressure resistant to a pressure of up to 100 bar. Such a pressure resistance is required to be able to work under the conditions required for hydropyrolysis. This means, that in order to perform hydropyrolysis, particularly the at least two locking systems on either side of the middle pyrolysis zone should be pressure resistant to a pressure of up to 100 bar.

Preferably, the closed pyrolysis zone is temperature resistant to a temperature of at least 450° C. This means, that particularly the at least two locking systems on either side of the middle pyrolysis zone are preferably temperature resistant to a temperature of at least 450° C.

Preferably, the apparatus comprises a pre-treatment zone located upstream from the pyrolysis zone and separated from the pyrolysis zone by a pyrolysis zone entry locking system. Thus, the pyrolysis zone entry locking system is one of the at least two locking systems on either side of the middle pyrolysis zone.

Preferably, the apparatus further comprises a pre-treatment zone entry locking system. In this case, the pre-treatment zone may also be sealed from the environment and/or the other zones, by simultaneously closing the pyrolysis zone entry locking system and the pre-treatment zone entry locking system. In the pre-treatment zone, the container with feedstock may for example be pre-heated. This is especially advantageous in a case where the feedstock needs to be liquefied, optionally in a reaction medium and/or heat transfer agent such as molten salt. The feedstock and optionally salt may for example be loaded in the open container, after which the container may be loaded in the pre-treatment zone when the pre-treatment zone entry locking system is open and the pyrolysis zone entry locking system is closed. Subsequently closing the pre-treatment zone entry locking system creates a sealed environment in the pre-treatment zone.

Preferably, the apparatus comprises heating means, cooling means and/or pressurizing means configured to adjust the temperature and/or pressure of the pre-treatment zone independently from the other zones. In this way, the temperature of the pre-treatment zone may be set to a temperature which is lower than the temperature in the pyrolysis zone, such that liquefaction of the feedstock may be executed under suitable circumstances, i.e. at a temperature which is optimal for liquefaction, without pyrolyzing the feedstock. The pressure may for example be set to ambient during liquefaction, and increased to a pyrolysis pressure, e.g. 100 bar, before opening the pyrolysis zone entry locking system in order to ensure transfer of the container to the pyrolysis zone.

Preferably, the apparatus further comprises a pre-treatment carrier gas inlet and a pre-treatment carrier gas outlet, both in fluid communication with the pre-treatment zone. The pre-treatment carrier gas inlet is advantageously in fluid communication with a carrier gas source, such as one or more gas cylinders, for feeding carrier gas to the reactor. The carrier gas source may be the same as or different from the carrier gas source used for feeding gas to the pyrolysis zone. The pre-treatment carrier gas outlet may be in fluid communication with one or more condensers, in which any vapors which may be released by the feedstock in the pre-treatment zone may be captured as a liquid product.

Preferably, the apparatus comprises a cooling zone located downstream from the pyrolysis zone, and separated from the pyrolysis zone by a pyrolysis zone exit locking system. Thus, the pyrolysis zone exit locking system is one of the at least two locking systems on either side of the middle pyrolysis zone.

Preferably, the apparatus comprises cooling means configured to adjust the temperature cooling zone independently from the other zones. In the cooling zone, the container, and thus the feedstock in the container are preferably cooled to a temperature at which the container may be handled by hand. Thus, preferably the cooling means are configured to cool a container in the cooling zone from pyrolysis temperatures to a temperature below 50° C.

Preferably, the apparatus further comprises a cooling zone exit locking system located downstream from the pyrolysis zone exit locking system. When in the closed position while the pyrolysis zone exit locking system is also in the closed position, the container is easily held in place in the cooling zone. The cooling zone however is not required to be operable under the same high temperatures as the pyrolysis zone and pre-treatment zone, and therefore the cooling zone exit locking system need not meet the same demands with respect to temperature resistance as either of the pyrolysis zone locking systems. Any type of means suitable to provide a surface for the container to be positioned on top of, e.g. a less temperature and pressure resistant valve (e.g. up to 100° C. and 100 bar), will suffice.

It will be clear that a locking system between two zones in the apparatus may have two names: The pyrolysis zone entry locking system is the same as the pre-treatment zone exit locking system, and the pyrolysis zone exit locking system is the same as the cooling zone entry locking system.

Preferably, a maximum dimension of the apparatus is 2 m. Thus, either the length, height or width of the apparatus are less than 2 m. Preferably all of the length, height and width are less than 2 m. This makes the apparatus especially suitable for placing it in a research laboratory environment.

The method according to the invention comprises steps a)-f):
a) loading the feedstock into the open container,
b) placing the container in the pre-treatment zone,
c) pre-heating the feedstock, preferably to a temperature of 50-250° C.,
d) transferring the container to the pyrolysis zone,
e) pyrolyzing the feedstock, preferably at a temperature of 400-450° C. and a pressure of 1-60 Bar,
f) transferring the container to the cooling zone and allowing the feedstock to cool, preferably to a temperature of below 50° C.

Loading the feedstock into the open container may be done by any method, the simplest being scooping the feedstock into the open container. Preferably, the weight of the loaded feedstock is recorded. This may for example be realized by weighing the container before and after loading the feedstock and noting the difference.

Optionally, the feedstock may be loaded into the open container together with a reaction medium, a solid or liquid catalyst, and/or a heat transfer agent, such as an ionic liquid or molten salt which is liquid at least during the pyrolysis step e). Such an ionic liquid or molten salt may serve both as a catalyst and heat transfer agent. Preferably, the container is filled to a maximum of 50% of its volume, more preferably to a maximum of 30% of its volume, in order to account for volume changes and/or gas development upon heating. With the pre-treatment zone entry locking system in the open position and the pyrolysis entry zone locking system in the closed position, the container is then loaded into the pre-treatment zone. Optionally, the container is loaded together with a damping means, such as a stainless steel spring, to prevent breaking of the container (e.g., a quartz tube) on impact. Subsequently, the pre-treatment zone entry locking system is closed. The feedstock is then pre-heated in step c). Preferably, the atmosphere, and optionally the pressure are identical to the atmosphere in the pyrolysis step e). After pre-heating preferably about 10-20 minutes, the pyrolysis zone entry locking system is placed in the open position in step d), and the container is allowed to pass (c.q. fall) to the pyrolysis zone underneath, after which the pyrolysis zone entry locking system is placed in the closed position. The feedstock may then be pyrolyzed in step e) under the preferred pyrolysis conditions. After pyrolysis is complete, the pyrolysis zone exit locking system is placed in the open position and the container with pyrolyzed feedstock (char) is moved to the cooling zone, with the cooling zone exit locking system placed in the closed position. After placing the pyrolysis zone exit locking system in the closed position, the feedstock is allowed to cool down. When the temperature is sufficiently low, the cooling zone exit locking system is opened, and the container with char and the optional damping means are received.

It is preferred that step d) comprises opening a pyrolysis zone entry locking system before transferring and closing the pyrolysis entry zone locking system after transferring and that step f) comprises opening a pyrolysis zone exit locking system before transferring and closing a pyrolysis zone exit valve after transferring.

Preferably, step e) is performed under a full or partial hydrogen atmosphere for hydropyrolysis, preferably at a partial hydrogen pressure of between 1 and 100 bar, more preferably between 20 and 80 bar, even more preferably between 30 and 60 bar. These conditions are possible due to the pressure resistance of both pyrolysis zone locking systems.

In a preferred embodiment, the method comprises step g) collecting the pyrolyzed feedstock from the container, preferably followed by analysis of the pyrolyzed feedstock. This allows studying the influence of different settings (temperature of the different zones, type and amount of heat transfer medium, catalyst in the catalytic upgrading reactor) on the char composition. It is thus preferred that step g) also comprises opening a cooling zone exit locking system, collecting the container, and optionally closing the cooling zone exit locking system before collecting the pyrolyzed feedstock.

It is especially advantageous if all pyrolysis products are captured and analyzed. Therefore, preferably step e) further comprises collecting vapor pyrolysis products, preferably followed by analysis of the collected vapor pyrolysis products.

Even more preferably, the collected vapor pyrolysis products are catalytically upgraded, which may be executed in the catalytic upgrading reactor. In case of catalytic upgrading, the catalytic upgrading of the collected vapor pyrolysis products is preferably followed by analysis of the upgraded vapor pyrolysis products. The non-condensable gases may for example be analyzed by the on-line analyzer, such as a gas chromatograph, whereas the condensable gases may be collected, for example by the at least one condenser, and subsequently analyzed by suitable methods such as NMR, IR, GC-FID-MIS, CHN elemental analysis, H₂O content analysis, or any other suitable method to determine product properties and molecular composition.

It will be clear to the skilled person that the method according to the invention can also be executed semi-continuously, by sequentially opening and closing locking systems. For example, for the pre-treatment step of a container A, the pre-treatment zone entry locking system is first put in the open position and the pyrolysis entry zone locking system is in the closed position, after which the container A may be loaded into the pre-treatment zone. Subsequently, the pre-treatment zone entry locking system is closed, thereby securing container A in the pre-treatment zone for the pre-treatment step. After pre-treatment of container A, the pyrolysis zone entry locking system is opened, and container A moves to the pyrolysis zone. Closing the pyrolysis zone entry locking system secures container A in the pyrolysis zone for the pyrolysis step. After closing the pyrolysis zone entry locking system, the pre-treatment zone entry locking system may be opened for loading a container B into the pre-treatment zone, after which the pre-treatment zone entry locking system may be closed, thereby securing container B in the pre-treatment zone for the pre-treatment step. After pyrolysis of container A, the pyrolysis zone exit locking system may be opened, and container A transfers to the cooling zone. Sequentially closing the pyrolysis zone exit locking system and opening the pyrolysis zone entry locking system allows container B to pass to the pyrolysis zone. Then the pyrolysis zone entry locking system may be closed, and the pre-treatment zone locking system opened, and a container C may be placed into the pre-treatment zone, after which the pre-treatment zone entry locking system may be closed again, securing container C in the pre-treatment zone. After container A has cooled, it may be removed from the system by opening the cooling zone exit locking system. This completes a chain of zone movement of container A. Closing the cooling zone exit locking system puts the apparatus in position for completing the chain of zone movement of container B, and so on and so forth.

The feedstock may for example be any type of carbon containing solid waste which can be converted to fuels and chemicals, e.g., biomass and waste plastics. Preferably the feedstock comprises biomass, and preferably the biomass comprises lignin. The pyrolysis of these feedstocks has been especially cumbersome to study with the presently available pyrolysis research equipment, and the method of the present invention provides significant advantages for these feedstocks. In a further preferred embodiment, the feedstock comprises waste plastics.

### BRIEF DESCRIPTION OF THE FIGURES

FIG. 1 is a schematic overview of an embodiment of an apparatus according to the invention.

FIG. 2 is a detailed schematic overview of an embodiment of an apparatus according to the invention.

FIG. 3. is a graph of the product yield for examples using two types of biomass with different conditions: salts, pressure, and carrier gas.

FIG. 4 is a graph of the composition of the liquid product for lignin and sawdust pyrolysis with 10:90 biomass: salt ratio and without salts.

FIG. 5 is a graph of GPC traces for original lignin and lignin oils at atmospheric pressure with and without molten salts: 400° C., preheater 210° C.

FIG. 6 is a graph of the yields for high pressure experiments with N₂ and H₂ as carrier gas, 20 bar, 375° C. Biomass-Salt ratio 60:40.

FIG. 7 is a graph of the decrease of solid residue for increasing pyrolysis time of a plastic sample.

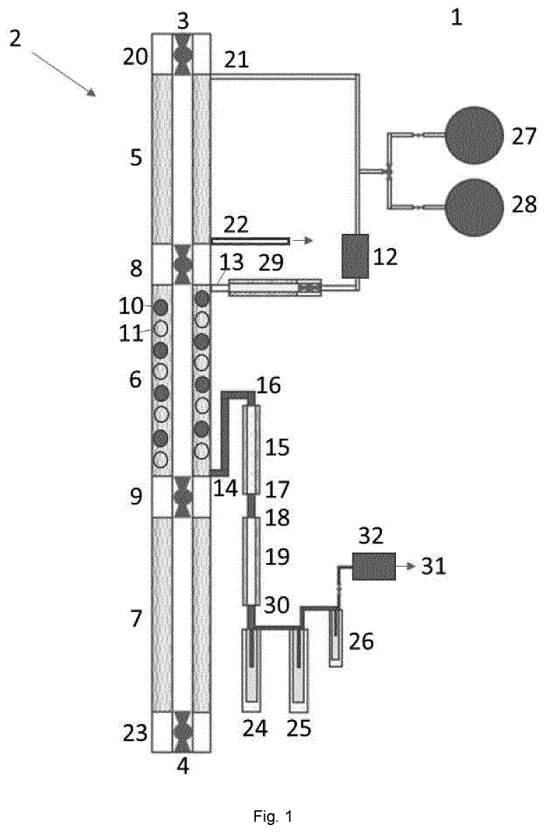

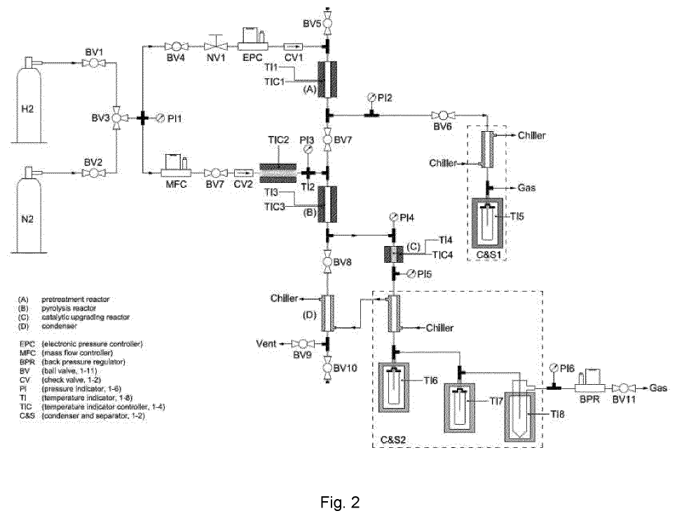

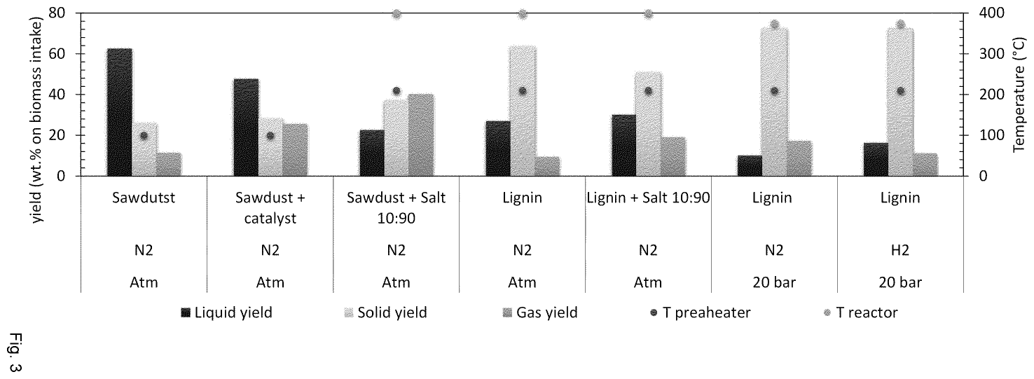

### DETAILED DESCRIPTION OF FIG. 1

FIG. 1 shows an apparatus 1 for pyrolyzing a feedstock in an open container. The apparatus comprises a downer 2 with an entry 3 for feeding and an exit 4 for removing the container from the downer 2. The downer 2 is divided in 3 zones, 5, 6, and 7, by locking systems 8 and 9. Locking systems 8 and 9 are configured to simultaneously close, such that the pyrolysis zone 6 may be sealed. When sealed, the pyrolysis zone is advantageously pressure resistant to a pressure of up to 100 bar, thereby making the apparatus suitable for hydropyrolysis. When the locking systems 8 and 9 are in an open position, the open container may pass through each of the individual locking zones from one zone to a downstream zone. For example, when locking system 8 is in an open position, the open container may be transferred from the pre-treatment zone 5 to pyrolysis zone 6, and when locking system 9 is in an open position, the open container may be transferred from the pyrolysis zone 6 to the cooling zone 7. The apparatus further comprises heating means 10, cooling means 11, and pressurizing means—for example comprising mass flow controller 12 and back pressure regulator 32—which are configured to adjust the temperature and pressure of at least the pyrolysis zone 6 independently from the other zones 5 and 7. Pre-treatment zone 5 is located upstream from the pyrolysis zone 6 and is separated from the pyrolysis zone by a pyrolysis zone entry locking system 20. Cooling zone 7 is located downstream from the pyrolysis zone 6 and is separated from the pyrolysis zone 6 by a pyrolysis zone exit locking system 9. At the downstream end of cooling zone 7, a cooling zone exit locking system 23 is located.

The apparatus 1 further comprises a pyrolysis carrier gas inlet 13 and a pyrolysis vapor outlet 14, both in fluid communication with the pyrolysis zone 6. A pre-heater 29 in fluid communication with the pyrolysis carrier gas inlet 13 serves to pre-heat the carrier gas. The apparatus further comprises a pre-treatment carrier gas inlet 21 and a pre-treatment carrier gas outlet 22, both in fluid communication with the pre-treatment zone 5.

The apparatus 1 further comprises a pyrolysis carrier gas inlet 21 and a pyrolysis carrier gas outlet 22, both in fluid communication with the pre-treatment zone 5. Carrier gas sources 27 and 28 are in fluid communication with both pyrolysis carrier gas inlets 13 and 21.

A catalytic upgrading reactor 15 has a gas inlet 16 in fluid communication with the pyrolysis vapor outlet 14. The catalytic upgrading reactor 15 may be a catalytic fixed-bed reactor. The catalytic upgrading reactor 15 comprises a gas outlet 17 in fluid communication with the inlet 18 of a cooling unit 19. The gas outlet of the cooling unit 19 may be connected to several condensers 24, 25, 26, in series, such that the condensable gases in a vapor stream leaving the gas outlet of the cooling unit 19 may be condensed before the vapor stream, which is stripped from condensable gases leaves through outlet 31. The apparatus may further comprise an analyser for analysing pyrolysis gas products. The apparatus may have a maximum width, height, and/or length of 2 m, for fitting in a laboratory.

In an example of a method according to the invention, a feedstock such as biomass, is loaded to an open container, such as a quartz tube. In order to prevent the feedstock from accidentally spilling from the container, the container may be closed with a perforated lid. Pre-treatment zone entry locking system 20 is placed in an open position, and pyrolysis zone entry locking system 8 is placed in the closed position, such that the container may pass through pre-treatment zone entry locking system and is held in the pre-treatment zone by the closed pyrolysis zone entry locking system 8. The pre-treatment zone entry locking system 20 may then be placed in the closed position, and carrier gas may be led through carrier gas inlet 21, leaving the pre-treatment zone through pre-treatment carrier gas outlet 22. Pre-treatment zone 5 is preferably heated to a temperature of 50-250° C. for pre-heating the feedstock. In case a heat-transfer medium is used, pre-heating may also serve for liquefaction of the feedstock in the heat-transfer medium. Suitably, the heat-transfer medium is a liquid salt. After pre-treatment, the pyrolysis zone entry locking system 8 is placed in the open position while pyrolysis zone exit locking system is placed in the closed position, causing the container to pass through the pyrolysis zone entry locking system 8, being held in the pyrolysis zone by the closed pyrolysis zone exit locking system 9. After closing pyrolysis zone entry locking system 8, the feedstock may be pyrolyzed, preferably at a temperature of about 400-450° C., maintained by the heating means 10 and cooling means 11, and a pressure of 1-60 bar, maintained by pressurizing means 12 and 32, of the pyrolysis carrier gas, which enters the pyrolysis zone 6 through pyrolysis carrier gas inlet 13, optionally pre-heated by pre-heater 29. The pyrolysis vapors leave the pyrolysis zone 6 through pyrolysis vapor outlet 16, and are upgraded in catalytic upgrading reactor 15, after which the upgraded pyrolysis vapors are cooled and condensed in cooler 19 and condensers 24, 25, and/or 26. Non-condensable gases may leave through outlet 31. After pyrolysis, the pyrolysis zone exit locking system 9 is placed in an open position, while cooling zone exit locking system 23 is placed in the closed position, causing the container to pass through the pyrolysis zone exit locking system 9, being held in the cooling zone 7 by closed cooling zone exit locking system 23. The pyrolysis zone exit locking system 9 is placed in the closed position, and the container with pyrolyzed feedstock is cooled, preferably to a temperature below 50° C., before leaving the apparatus through exit 4 when pyrolysis zone exit locking system 23 is placed in the open position.

### EXAMPLES

### Operation Description

The temperatures of the pre-treatment zone and pyrolysis zone are set, then the sample (about 5-15 g) is located in a stainless steel tube (outer diameter Ø18 mm×thickness 0.5 mm×length 350 mm) that is placed in the pre-heater (pre-treatment zone) for pre-heating for 10 min, which is then allowed to fall into the reactor (pyrolysis zone) where the sample is pyrolyzed for 20 min. Subsequently, the tube is allowed to fall into the cooler (cooling zone) where it stays for another 10 min to allow it to cool down. The pre-heater, reactor, and cooler each are made from a stainless steel tube of Ø24.5 mm×thickness 2 mm×400 mm length. Then the tube is removed from the cooler (cooling zone) and the solids are collected from the tube. The pyrolysis vapors pass by a set of condensers of which the first one is set at 0° C. and the second at −20° C. The liquid in both condensers is accumulated, collected, and weighted. For the examples in this document all the experiments were carried out with a carrier gas flow of 17 ml/min. The solid yield is obtained by determining the weight from the solid remained in the tube after the process. In the case of pyrolysis in molten salts, it is assumed that all the salts remain in the tube. For the liquid yield, all the condensers are weighed before and after the process to obtain the yield, the moisture recovered from the pre-treatment zone is added to the water phase yield.

$$Liquid yield = \frac{{W}_{condenser tubes} - {W}_{empty condenser tubes}}{{W}_{b i o m a s s}} \times 100$$

$$Char yield = \frac{{W}_{tubes after reaction} - {W}_{empty reaction tubes}}{{W}_{b i o m a s s}} \times 100$$

$$Gas yield = 100 - Liquid yield - Char yield$$

### Materials Used

Pinewood sawdust (BEMAP-pine nr B03) was supplied by Bemap Houtmeel BV. Indulin-AT is a purified pine Kraft lignin. It was supplied by MWV specialty chemicals. It does not contain hemicellulosic material. It has an ash content of 3 wt %. Nitrogen gas was obtained from Linde (>99.99% purity). Tetrahydrofuran (THF, anhydrous, ≥99.9%, with 250 ppm BHT as inhibitor), and di-n-butyl ether (DBE) were obtained from Sigma-Aldrich and were reagent grade (>99% purity). ZnCl₂, NaCl, and KCl (analytical purity >99%) were purchased from VWR Chemicals (Belgium). Toluene was supplied by Biosolve Chimie. A granular alumina bonded H-ZSM-5 catalyst was obtained from Yangzhou Baisheng Catalyst Co., Ltd., PR China. H-ZSM-5/Al₂O₃ had a 60/40 wt % ratio and the molar ratio of SiO₂/Al₂O₃ was 28. The catalyst was crushed and sieved to achieve a particle size between 0.5 mm and 1 mm.

TABLE 1
Conditions and product yields for the experiments carried out as examples.

| | | | | | | | Liquid | Solid | Gas |
|---|---|---|---|---|---|---|---|---|---|
| | | Carrier | | T (° C.) | T (° C.) | T (° C.) | yield | yield | yield |
| Pressure | | gas | Feedstock | preheater | reactor | cat. bed | (%) | (%) | (%) |
| 1 | bar | N₂ | Sawdust | 100 | 475 | — | 62.6 | 26 | 11.4 |
| 1 | bar | N₂ | Sawdust + | 100 | 475 | 450 | 47.7 | 28.2 | 25.6 |
| | | | catalyst | | | | | | |
| 1 | bar | N₂ | Sawdust + | 210 | 400 | — | 22.6 | 37.2 | 40.2 |
| | | | Salt 10:90 | | | | | | |
| 1 | bar | N₂ | Lignin | 210 | 400 | — | 27 | 63.7 | 9.4 |
| 1 | bar | N₂ | Lignin + | 210 | 400 | — | 30.1 | 50.9 | 19 |
| | | | Salt 10:90 | | | | | | |
| 20 | bar | N₂ | Lignin + | 210 | 375 | — | 10.1 | 72.7 | 17.2 |
| | | | Salt 60:40 | | | | | | |
| 20 | bar | N₂ | Lignin + | 210 | 375 | — | 16.3 | 72.6 | 11.1 |
| | | | Salt 60:40 | | | | | | |

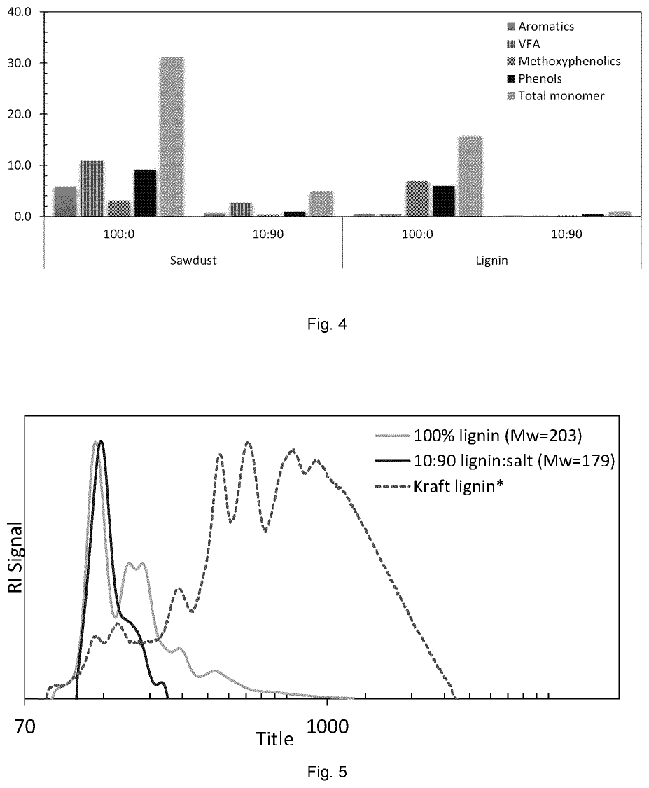

The yields are also graphically represented in FIG. 4. Liquids were analyzed using GC×GC-FID and GPC techniques. The samples were prepared by diluting the liquid samples in THF about 30 times, filtering them and injecting them in the analysis facility, GC×GC-FID gives the composition quantified per functional group (FIG. 4).

For the GPC, FIG. 5 shows the depolymerization degree of the lignin oils after pyrolysis, the molecular weight distribution is compared between feedstock and products and then see the effect of each experimental condition in the final product.

### High Pressure Operation

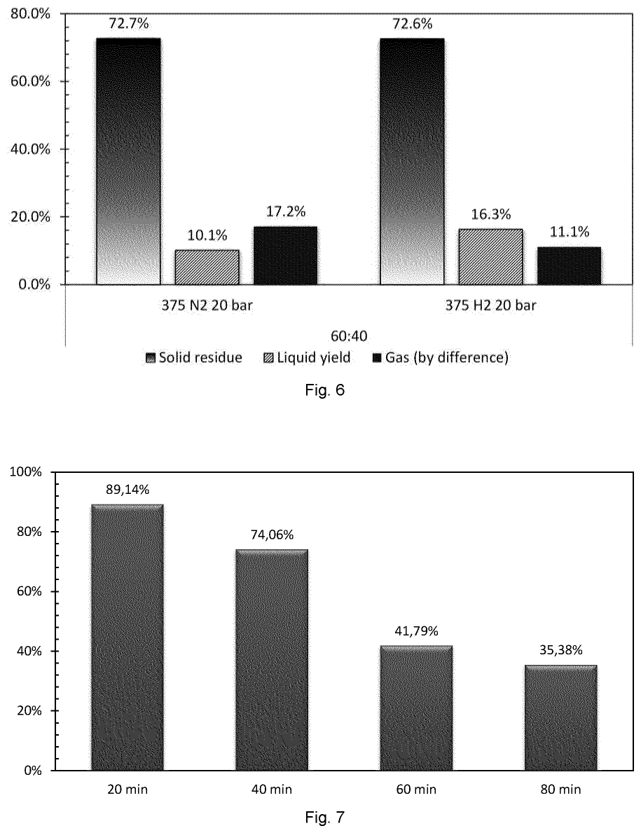

The temperatures of the pre-treatment zone and pyrolysis zone are set, the system pressure is set, the pre-treatment zone pressure is released to atmospheric pressure, then the tube with the sample is placed and sealed (by closing both pre-treatment zone locking systems) in the pre-treatment zone. While the pressure in this section reaches the pre-set pressure, the tube stays in the pre-treatment section for a period of 10 min, then falls into the pyrolysis zone wherein it is sealed and stays for 20 min. During the reaction time the pressure in the system is kept constant by a small flow of the carrier gas and a back pressure regulator valve. The tube is subsequently dropped and sealed in the cooling zone after which the pressure in the cooling zone is gradually released to a safe value (˜1 bar). The temperature of the cooling zone is around room temperature or lower, and the tube is kept in the cooling zone for about 10 min after which it is removed from the system and the solids are collected from the tube. During the reaction time the pyrolysis vapors pass by a set of condensers of which the first one is set at 0° C., and the second at −20° C. The liquid in both condensers was accumulated, collected and weighed (see FIG. 6).

### Plastic Sample

In the case of pyrolysis of a polymer, a sample of an unknown blend of polymers was pyrolyzed in the unit, the sample was placed in the pre-treatment zone which was set at 100° C. and left there for 1 min before dropping it to the reactor at 500° C. The reaction time was increased with each injection while the liquid samples were collected only once. The total liquid yield for all the injections was 8.01 wt. % on the feedstock input (see FIG. 7).

TABLE 2
Solid residence time effect in the solid
residue yield, conversion time example.

| Solid | Solid residue |
|---|---|
| residence | yield (wt. % on |
| time (min) | feedstock input) |
| 20 | 89.14 |
| 40 | 74.06 |
| 60 | 41.79 |
| 80 | 35.38 |

### Liquid Samples were Analyzed with GC-MS and GC×GC-FID

TABLE 3
Retention times and compounds of liquid yield. Tᵣ = 500° C.

| Retention | |
|---|---|
| time (s) | Compound name |
| 1.88 | 1-Pentene, 2-methyl- |
| 1.97 | 2-Pentene, 4-methyl-, (Z)- |
| 2.10 | Furan, tetrahydro- |
| 2.23 | 2,4-Hexadiene, (Z,Z)- |
| 2.59 | 1,5-Hexadiene, 2-methyl- |
| 2.85 | 3-Heptene, (E)- |
| 3.04 | Cyclopropane, trimethylmethylene- |
| 3.37 | 1,4-Hexadiene, 4-methyl- |
| 3.58 | 1,3,5-Hexatriene, 3-methyl-, (Z)- |
| 3.64 | 1,4-Cyclohexadiene, 1-methyl- |
| 3.77 | 2,4-Dimethyl-1-hexene |
| 4.14 | Heptane, 4-methyl- |
| 4.97 | Cyclopentene, 1,2,3-trimethyl- |
| 5.21 | trans-3,5-Dimethylcyclohexene |
| 5.29 | 1,4-Heptadiene, 3-methyl- |
| 5.66 | 1,3-Cyclohexadiene, 5,6-dimethyl- |
| 6.02 | Bicyclo[3.1.0]hexane, 1,5-dimethyl- |
| 6.20 | Cyclohexane, 1-methyl-4-methylene- |
| 6.50 | Spiro[2.4]heptane, 4-methylene- |
| 6.74 | 2,4-Dimethyl-1-heptene |
| 6.89 | 1,2-Dimethyl-1,4-cyclohexadiene |
| 7.20 | 1,1-Dimethyl-4-methylenecyclohexane |
| 7.35 | Cyclohexane, 1,3,5-trimethyl-, (1.alpha.,3.alpha.,5.beta.)- |
| 7.92 | p-Xylene |
| 10.58 | 3,3-Dimethyl-6-methylenecyclohexene |
| 10.78 | 1,6-Dimethylhepta-1,3,5-triene |
| 12.82 | Benzene, 1,3,5-trimethyl- |
| 23.88 | 3-Tridecene, (Z)- |
| 26.93 | 1H-Indene, 1,1-dimethyl- |
| 37.73 | Butylated Hydroxytoluene |
| 47.61 | (+−)-3-(2-Carboxy-trans-propenyl)-2,2- |
| | dimethylcyclopropane-trans-1-carboxylic |
| | acid, [1.alpha., 3.beta.(E)]- |

The invention furthermore relates to the following clauses:
1. Apparatus for pyrolyzing a feedstock in an open container, the apparatus comprising a downer with an entry for feeding and an exit for removing the container from the downer, the downer being divided into at least three zones by at least two locking systems on either side of a middle pyrolysis zone, wherein the at least two locking systems are configured to simultaneously close for sealing the pyrolysis zone, wherein the at least two locking systems are further configured to let through the container in an open position, the apparatus comprising heating means, cooling means and pressurizing means configured to adjust the temperature and pressure of at least the pyrolysis zone independently from the other zones.
2. Apparatus according to clause 1, further comprising a pyrolysis carrier gas inlet and a pyrolysis vapor outlet, both in fluid communication with the pyrolysis zone.
3. Apparatus according to clauses 2, comprising a pre-heater in fluid communication with the pyrolysis carrier gas inlet.
4. Apparatus according to clause any one of the preceding clauses, further comprising a catalytic upgrading reactor having a gas inlet in fluid communication with the pyrolysis vapor outlet.
5. Apparatus according to clause 3 or 4, wherein the catalytic upgrading reactor is a moving bed reactor, fluidized bed reactor, circulating fluidized bed reactor or a fixed-bed reactor, preferably a fixed-bed reactor.
6. Apparatus according to clause 4 or 5, wherein the catalytic upgrading reactor comprises a gas outlet in fluid communication with the inlet of a cooling unit.
7. Apparatus according to any one of the preceding clauses, wherein the closed pyrolysis zone is pressure resistant to a pressure of up to 100 bar.
8. Apparatus according to any one of the preceding clauses, comprising a pre-treatment zone located upstream from the pyrolysis zone and separated from the pyrolysis zone by a pyrolysis zone entry locking system.
9. Apparatus according to clause 8, further comprising a pre-treatment zone entry locking system.
10. Apparatus according to clause 9, further comprising a pre-treatment carrier gas inlet and a pre-treatment carrier gas outlet, both in fluid communication with the pre-treatment zone.
11. Apparatus according to any one of the preceding clauses, comprising a cooling zone located downstream from the pyrolysis zone, and separated from the pyrolysis zone by a pyrolysis zone exit locking system.
12. Apparatus according to clause 11, further comprising a cooling zone exit locking system located downstream from the pyrolysis zone exit locking system.
13. Apparatus according to any one of the preceding clauses, further comprising an analyser for analysing pyrolysis gas products.
14. Apparatus according to any one of clauses 1-13, wherein a maximum dimension of the apparatus is 2 m.
15. Method for pyrolyzing a feedstock in an open container, the method comprising:
   a) loading the feedstock into the open container,
   b) placing the container in a pre-treatment zone,
   c) pre-heating the feedstock, preferably to a temperature of 50-250° C.,
   d) transferring the container to a pyrolysis zone,
   e) pyrolyzing the feedstock, preferably at a temperature of 400-450° C. and a pressure of 1-60 bar,
   f) transferring the container to a cooling zone and allowing the feedstock to cool, preferably to a temperature of below 50° C.
16. Method according to clause 15, further comprising
   g) collecting the pyrolyzed feedstock from the container, preferably followed by analysis of the pyrolyzed feedstock.
17. Method according to clause 15 or 16, wherein step e) is performed under a full or partial hydrogen atmosphere for hydropyrolysis, preferably at a partial hydrogen pressure of between 1 and 100 bar, more preferably between 20 and 80 bar, even more preferably between 30 and 60 bar.
18. Method according to any one of clauses 15-17, wherein step e) further comprises collecting vapor pyrolysis products, preferably followed by analysis of the collected vapor pyrolysis products or by catalytic upgrading of the collected vapor pyrolysis products, the catalytic upgrading of the collected vapor pyrolysis products preferably followed by analysis of the upgraded vapor pyrolysis products.
19. Method according to any one of clauses 15-18, wherein the feedstock comprises biomass, preferably wherein the biomass comprises lignin.
20. Method according to any one of clauses 15-18, wherein the feedstock comprises waste plastics.
21. Method according to any one of clauses 15-20, wherein step d) comprises opening a pyrolysis zone entry locking system before transferring and closing the pyrolysis entry zone locking system after transferring and wherein step f) comprises opening a pyrolysis zone exit locking system before transferring and closing a pyrolysis zone exit valve after transferring.
22. Method according to any one of clauses 15-21, wherein the method is executed semi-continuously.

1. An apparatus for pyrolyzing a feedstock in an open container, the apparatus comprising a downer reactor with an entry located at the top end of the downer for feeding and an exit located at the bottom end of the downer for removing the container from the downer, the downer being divided into at least three vertically arranged zones by at least two locking systems on either side of a middle pyrolysis zone, wherein the at least two locking systems are configured to simultaneously close for sealing the pyrolysis zone, wherein the at least two locking systems are further configured to let through the container in an open position, the apparatus comprising heating means, cooling means and pressurizing means configured to adjust the temperature and pressure of at least the pyrolysis zone independently from the other zones.

2. The apparatus according to claim 1, further comprising a pyrolysis carrier gas inlet and a pyrolysis vapor outlet, both in fluid communication with the pyrolysis zone.

3. The apparatus according to claim 2, comprising a pre-heater in fluid communication with the pyrolysis carrier gas inlet.

4. The apparatus according to claim 1, further comprising a catalytic upgrading reactor having a gas inlet in fluid communication with the pyrolysis vapor outlet, preferably wherein the catalytic upgrading reactor is a moving bed reactor, fluidized bed reactor, circulating fluidized bed reactor or a fixed-bed reactor, more preferably a fixed-bed reactor.

5. The apparatus according to claim 1, wherein the apparatus is suitable for pyrolyzing feedstock on a gram-scale, preferably 1-100 g.

6. The apparatus according to claim 4, wherein the catalytic upgrading reactor comprises a gas outlet in fluid communication with the inlet of a cooling unit.

7. The apparatus according to claim 1, wherein the closed pyrolysis zone is pressure resistant to a pressure of up to 100 bar.

8. The apparatus according to claim 1, comprising a pre-treatment zone located upstream from the pyrolysis zone and separated from the pyrolysis zone by a pyrolysis zone entry locking system.

9. The apparatus according to claim 8, further comprising a pre-treatment zone entry locking system.

10. The apparatus according to claim 9, further comprising a pre-treatment carrier gas inlet and a pre-treatment carrier gas outlet, both in fluid communication with the pre-treatment zone.

11. The apparatus according to claim 1, comprising a cooling zone located downstream from the pyrolysis zone, and separated from the pyrolysis zone by a pyrolysis zone exit locking system.

12. The apparatus according to claim 11, further comprising a cooling zone exit locking system located downstream from the pyrolysis zone exit locking system.

13. The apparatus according to claim 1, further comprising an analyser for analysing pyrolysis gas products.

14. The apparatus according to claim 1, wherein a maximum dimension of the apparatus is 2 m.

15. A method for pyrolyzing a feedstock in an open container, the method comprising:
a) loading the feedstock into the open container,
b) placing the container in a pre-treatment zone,
c) pre-heating the feedstock, preferably to a temperature of 50-250° C.,
d) transferring the container to a pyrolysis zone by gravity,
e) pyrolyzing the feedstock, preferably at a temperature of 400-450° C. and a pressure of 1-60 bar,
f) transferring the container to a cooling zone by gravity and allowing the feedstock to cool, preferably to a temperature of below 50° C.

16. The method according to claim 15, further comprising
g) collecting the pyrolyzed feedstock from the container, preferably followed by analysis of the pyrolyzed feedstock.

17. The method according to claim 15, wherein step e) is performed under a full or partial hydrogen atmosphere for hydropyrolysis, preferably at a partial hydrogen pressure of between 1 and 100 bar, more preferably between 20 and 80 bar, even more preferably between 30 and 60 bar.

18. The method according to claim 15, wherein step e) further comprises collecting vapor pyrolysis products, preferably followed by analysis of the collected vapor pyrolysis products or by catalytic upgrading of the collected vapor pyrolysis products, the catalytic upgrading of the collected vapor pyrolysis products preferably followed by analysis of the upgraded vapor pyrolysis products.

19. The method according to claim 15, wherein the feedstock comprises biomass, preferably wherein the biomass comprises lignin.

20. The method according to claim 15, wherein the feedstock comprises waste plastics.

21. The method according to claim 15, wherein step d) comprises opening a pyrolysis zone entry locking system before transferring and closing the pyrolysis entry zone locking system after transferring and wherein step f) comprises opening a pyrolysis zone exit locking system before transferring and closing a pyrolysis zone exit valve after transferring.

22. The method according to claim 15, wherein the method is executed semi-continuously.
